(19) United States
(12) Patent Application Publication
(10) Pub. No.: US 2003/0072776 A1
(43) Pub. Date: Apr. 17, 2003

(54) Emulsifiable concentrate compositions with silicone antifoams

(76) Inventors: Jinxia Sun, Hopewell Junction, NY (US); Charles Scott, Poughquag, NY (US); Robert E. Ruckle, Brewster, NY (US); George A. Policello, Ossining, NY (US)

Correspondence Address: CROMPTON CORPORATION, BENSON ROAD, MIDDLEBURY, CT, 06749, US

(21) Appl. No.: 09/905,844

(22) Filed: Jul. 13, 2001

Related U.S. Application Data
(63) Continuation-in-part of application No. 09/478,589, filed on Jan. 6, 2000, now abandoned.

(51) Int. Cl. C09K 3/00; A61K 7/00; A01N 25/00

(52) U.S. Cl. 424/401; 516/117; 504/363

(57) ABSTRACT

A foam control agent for emulsifiable concentrates and its use therein is disclosed wherein the agent comprises a soluble organosilicone of the formula:

(R²(R¹)₂SiO_{1/2})ₑ(SiO_{4/2})_{f}(SiO_{3/2}R¹)_{g}(SiO_{2/2})(R¹)₂)ₓ(SiO_{2/2}R¹Z)_{y}

wherein: e=2f+g+2; f=0 to 2; g=0 to 2; x=30 to 150; y=0 to 30; where, when y>0, x/y=4 to 18 and x+y≧30, and when y=0, the ratio x/e is in the range of from about 20/1 to about 10/1; R¹=alkyl of 1 to 18 carbon atoms; R²═R¹ or Z, provided that when y=0, R²═Z; Z═R³OBₙG; R³ is a divalent bridging group of two to four carbons; B is an alkylene oxide group containing two to four carbon atoms, wherein less than ten mole percent of the alkylene oxide functionality is ethylene oxide; n=8 to 30; and G=hydrogen, a hydrocarbon radical of 1 to 18 carbon atoms, or acetyl.

### CROSS REFERENCE TO RELATED APPLICATION

This application is a continuation-in-part of co-pending application Ser. No. 09/478,589 filed Jan. 6, 2000.

### BACKGROUND OF THE INVENTION

1. Field of the Invention

The present invention relates to foam control agents. More particularly, the present invention relates to a foam control agent that is a homogeneous mixture (i.e., one phase) in emulsifiable concentrate (EC) pesticidal compositions.

2. Description of Related Art

Emulsifiable concentrates are one of the most widely used pesticide formulation types for agricultural applications, because of its easy preparation and handling and ability to deliver higher poundage of pesticide active(s) effectively and economically. Emulsifiable concentrates are prepared from oily pesticide active ingredients or from low-melting solid active ingredients that can be dissolved in nonpolar hydrocarbon solvents such as xylene, C₉—C₁₂ aromatic solvents, or kerosene. Surfactant emulsifiers are added to these formulations to ensure spontaneous emulsification with good emulsion stability in the spray tank. A typical EC formulation contains approximately 20 percent to 80 percent active ingredient, 20 percent to 75 percent solvent, and 5 percent to 10 percent emulsifier. These formulations are diluted in water prior to use to form an emulsion.

Antifoams or defoamers frequently are used to control foam formation in pesticide processes or tank-side applications. Most conventional antifoams consist of mixtures of insoluble oil and hydrophobic solid particles. The EC formulation containing this type of antifoam is heterogeneous (i.e., the system consists of two insoluble phases). However, the heterogeneous phase is unstable, and eventually separation will occur during either the formulation or storage period, which causes cream problems. So far, this problem has not been solved in pesticide EC formulations.

U.S. Pat. No. 5,658,852 discloses that linear alkylsilicone compounds of a given formula or cyclic alkylsilicone compounds of a second given formula are adjuvants for agricultural applications of oil-containing compositions. Especially preferred alkylsilicone compounds have a degree of polymerization of ≦6 siloxane units and an alkyl content of ≧50 percent by weight. The compounds potentiate spreading of mineral or vegetable oils or oil-containing emulsions in dormant spray oils, crop oil concentrates, pesticides, and the like on difficult-to-wet surfaces, such as waxy leaf cuticles and arthropod exoskeletons.

U.S. Pat. No. 5,968,872 discloses that silicone polyether copolymers with a low degree of polymerization (3 to 4 Si units) provide control for foams generated by organosilicone based surfactants in water without the need for an additional antifoam component. The copolymer foam control agent is a compound of the formula (CH₃)₃SiO{SiO(CH₃)₂}_{X}(SiOCH₃R¹)_{Y}Si(CH₃)₃ wherein X+Y=1 to 2, Y=1, R¹ is an alkyleneoxide group of the formula CₐH₂ₐO(C₃H₆O)^{z}R² wherein a=3 to 4, z is 1 to 15, R² is hydrogen, a hydrocarbon radical of one to four carbons or acetyl. Compositions are provided for use in agricultural formulations that give improved foam control for foams generated from organosilicone surfactants in aqueous systems. The copolymer foam control agent is soluble in a surfactant matrix comprised of an organosilicone surfactant alone or in mixtures with short chain conventional surfactants. Clear, homogeneous, low foaming agricultural formulations are provided having a water insoluble copolymer foam control agent, and a water soluble or dispersible trisiloxane or tetrasiloxane alkoxylate when the formulation is used in an aqueous mixture.

### SUMMARY OF THE INVENTION

The present invention relates to a foam control agent that is a homogeneous mixture (i.e., one phase) in EC pesticidal compositions. The EC detailed here comprises, in general, an organic solvent, soluble organic pesticide or mixtures of organic solvent soluble organic pesticides, emulsifiers, and an organosilicone foam control agent that is soluble in the EC matrix.

More particularly, the present invention is directed to a foam control agent for emulsifiable concentrates comprising a soluble organosilicone of the formula:

(R²(R¹)₂SiO_{1/2})ₑ(SiO_{4/2})_{f}(SiO_{3/2}R¹)_{g}(SiO_{2/2}(R¹)₂)ₓ(SiO_{2/2}R¹Z)_{y}

wherein:

e=2f+g+2;

f=0 to 2;

g=0 to 2;

x=30 to 150;

y=0 to 30;

where, when y>0, x/y=4 to 18 and x+y≧30, and when y=0, the ratio x/e is in the range of from about 20/1 to about 10/1;

R¹=alkyl of 1 to 18 carbon atoms;

R²═R¹ or Z, provided that when y=0, R²═Z;

Z═R³OBₙG;

R³ is a divalent bridging group of two to four carbons;

B is an alkylene oxide group containing two to four carbon atoms, wherein less than ten mole percent of the alkylene oxide functionality is ethylene oxide;

n=8 to 30; and

G=hydrogen, a hydrocarbon radical of 1 to 18 carbon atoms, or acetyl.

In another embodiment, the present invention is directed to an emulsifiable concentrate comprising:

A) at least one soluble organosilicone of the formula:

{R²(R¹)₂SiO_{1/2}}ₑ{SiO_{4/2}}_{f}{SiO_{3/2}R¹}_{g}{SiO_{2/2}(R¹)₂}ₓ{SiO_{2/2}R¹Z}_{y}

wherein:

e=2f+g+2;

f=0 to 2;

g=0 to 2;

x=30 to 150;

y=0 to 30;

where, when y>0, x/y=4 to 18 and x+y≧30, and when y=0, the ratio x/e is in the range of from about 20/1 to about 10/1;

R¹=alkyl of 1 to 18 carbon atoms;

R²═R¹ or Z, provided that when y=0, R²═Z;

Z═R³OBₙG;

R³ is a divalent bridging group of two to four carbons;

B is an alkylene oxide group containing two to four carbon atoms, wherein less than ten mole percent of the alkylene oxide functionality is ethylene oxide;

n=8 to 30; and

G=hydrogen, a hydrocarbon radical of 1 to 18 carbon atoms, or acetyl;

B) at least one active ingredient; and

C) at least one emulsifier.

### DESCRIPTION OF THE PREFERRED EMBODIMENTS

The present invention is directed to a soluble organosilicone foam control agent having the structure

(R²(R¹)₂SiO_{1/2})ₑ(SiO_{4/2})_{f}(SiO_{3/2}R¹)_{g}(SiO_{2/2}(R¹)₂)ₓ(SiO_{2/2}R¹Z)_{y}

wherein:

e=2f+g+2;

f=0 to 2, preferably 0;

g=0 to 2, preferably 0;

x=30 to 150, preferably 60 to 100;

y=0 to 30, preferably 4 to 15;

where, when y>0, x/y=4 to 18, preferably 7 to 12, and x+y≧30; and when y=0, then x/e is 20/1 to 10/1, preferably 18/1 to 12/1;

R¹=alkyl of 1 to 18 carbon atoms; R²═R¹ or Z with the proviso that when y=0, R²═Z;

Z═R³OBₙG;

R³ is a divalent bridging group of two to four carbons, preferably C₃;

B is an alkylene oxide group containing two to four carbon atoms (i.e., ethylene oxide, propylene oxide, butylene oxide), wherein less than ten mole percent of the alkylene oxide functionalities are ethylene oxide, but preferably, none is ethylene oxide;

n=8 to 30, preferably 8 to 15; and

G=hydrogen, a hydrocarbon radical of 1 to 18 carbon atoms, or acetyl, preferably hydrogen.

The foam control agents of the present invention are useful in emulsifiable concentrates and, when so used, are preferably present at a concentration that delivers from about 1 ppm to about 200 ppm foam control agent to the spray mixture.

In addition to the foam control agents of the present invention, such emulsifiable concentrates will normally contain at least one active ingredient and at least one emulsifier. Further, one or more solvents may be present, as well as other optional ingredients.

Active Ingredients The choice of active(s) is predetermined by the intended application. The active ingredient includes any organic solvent soluble pesticide or mixture of pesticides. The liquid herbicides useful herein include, but are not limited to, chloroacetamides (dimethanamid) or chloroacetanilides, such as 2′-ethyl-6′-methyl-N-(ethoxymethyl)-2-chloroacetylanilide (acetochlor), 2′,6′-diethyl-N-propoxyethyl-2-chloroacetanilide (pretilachlor), 2-ethyl-6-methyl-N-(1′-methyl-2′-methoxyethyl)chloroacetylanilide (metachlor), N-butoxy methyl-2′-6′-diethyl-2-chloro acetylanilide (butachlor), and others, including alachlor, metolachlor, diethatyl, metazachlor, dimethachlor, and preferably acetochlor; dichloroacetamide (dichlormid); butylates; cinmethylin; aryloxyphenoxys, including fluazifop (e.g., fluazifop-P aryloxyphenoxy propionate); phenoxys and phenoxy derivatives, such as phenoxy esters, phenoxyalkanoic acids, phenoxyacetic acid, including 2,4-D ester; thiocarbamates or carbamothioates, including butylate, cycloate, molinate, pebulate, thiobencarb, triallate, vernolate, s-ethyl dipropyl carbamothioate, s-ethyl diethylcarbamothioate (ethiolate); dietholate; cyclohexanedione or cyclohexanone, including sethoxydim or clethodim; dinitroaniline (isopropalin); oximes (fluxofenim); aldehydes (acrolein); and the like.

Examples of useful solid herbicides include benzoic acids and derivatives of benzoic acid including 3,6-dichloro-o-anisic acid, also referred to by various trade or other names including naptalam and dicamba sold under various trade designations, including BANVEL, BANFEL, BANVEL CST, BANVEL D, BANVEL XG, MEDIBEN, BANEX, and the like, and irnidazolinyl benzoic acids and esters and salts thereof; ureas, such as tebuthiuron and AC 263,222; imines, such as CGA-248757; imides, including N-phenylphthalimide, such as Flumichlorac; phenoxys and derivatives of phenoxys, including phenoxyalkanoic acids or phenoxy acetic acids, such as 2,4-D (2,4-dichlorophenoxy acetic acid), 3,4-DA (3,4-dichlorophenoxy acetic acid), 2,4-DB (4-(2,4-dichlorophenoxy)acetic acid), 3,4-DB (4-(3,4-dichlorophenoxy)butanoic acid), 2,4-DEB (2-(2,4-dichlorophenoxy)ethyl benzoate), 2,4-DEP (tris{2-(2,4-dichlorophenoxy)ethyl}phosphite), MCPA acid (4-chloro-2-methylphenoxy acetic acid), MCPB acid (4-(4-chloro-2-methylphenoxy)butanoic acid), mecoprop, diclofop, difenopenten, and dichlorprop; aryloxyphenoxy propionates, such as fenoxaprop, haloxyfop, and Quizalofop-P; benzothiazoleacetic acids, such as benazolin; dimethyl tetrachloroterephthalate (DCPA); amides and derivatives of amides, such as pronamide, propanil and napropamide; acetamides, such as mefluidide; benzamides, such as isoxaben; chloroacetamides or chloroacetanilides, such as propachlor; dichloroacetamides, such as R-29148; dinitroanilines, such as benefin, oryzalin and prodiamine; nitriles, including benzonitriles and hydroxybenzonitriles, such as bromoxynil, ioxynil, and dichlobenil; diphenylethers or nitrodiphenylethers, such as acifluorfen, bifenox, fluoroglycofen, fomesafen, and oxyfluorfen; dithiocarbamates, such as metham; carbamates, including phenylcarbamates, such as desmedipham, phenmedipham, and asulam; ureas including phenylureas, such as fluometuron, linuron, siduron, and diuron; pyridazinonones, such as norflurazon and pyrazon; pyridines, such as dithiopyr and thiazopyr; pyridinones, such as fluridone; pyridinecarboxylic acids or picolinic acids, such as triclopyr, clopyralid, and picloram; oxadiazolidines, such as methazole; benzothiadiazoles, such as bentazon; and the like.

Liquid insecticides useful herein include, but are not limited to, organophosphates, such as fonofos (Dyfonate7), diazinon, malathion, parathion, and the like; piperonyl butoxides; synthetic pyrethroids, such as bioresmethrin, resmethrin, and zeta-cypermethrin; a synthetic pyrethrin-type, such as allethrin; and the like.

Solid insecticides include, but are not limited to, pyrethrins; carbamates, such as aldicard; synthetic pyrethroids, such as bifenthrin, permethrin, and tefluthrin; organophosphates, such as methyl parathion; organophosphates, such as acephate and dimethoate; organochlorines, such as endosulfan; carbamates, such as carbaryl(1-naphthyl N-methylcarbamate) and methomyl (s-methyl N-{methylcarbamoyl)oxy}-thioacetamimidate); and the like.

Fungicides include categories of aliphatic nitrogen fungicides; anilides; aromatic fungicides, including aromatic nitrites; benzimidazoles and benzimidazole precursors; carbamates; benzimidazole carbamates; chlorophenyls; conazoles; dicarboximides; dinitrophenyls; thiocarbamates and dithiocarbamates; imidazoles; morpholines; organo phosphorus fungicides; guanidines; hydroxyanilides; morpholines; oils, phenylamides; phenylsulfamides; phenylureas; pyridines; phthalimides; pyrimidinamines; pyrimidines; pyrimidinols; quinolines; quinones; quinoxalines; thiazoles; stobilurins; triazoles; xylylanines; various unclassified fungicides; and the like.

Examples of liquid fungicides useful herein include, but are not limited to, morpholines, such as fenpropidin, fenpropimorph, and tridermorph; piperalin; conazoles, such as flusilazole, propiconazole, tebuconazole, triadimefon, and the like; pyridines, such as pyrifenox; thiazoles, such as etradiazole; organo phosphorus compounds, such as phosdiphen; imidazoles, such as pefurazoate; and the like.

Blends of pesticide may be used.

Preferably, the amount of total pesticide in the emulsifiable concentrate is between about 2 percent to about 97 percent by weight, more preferably about 50 percent to about 90 percent by weight, and most preferably about 65 percent to about 80 percent by weight.

Emulsifiers

The emulsifiers of the emulsifiable concentrates of the present invention comprise at least one emulsifier, which can be a nonionic surfactant, an ionic surfactant, or a blend of both type of surfactants. The useful nonionic surfactants include alkoxylated block polymers, alkoxylated alcohols, alkoxylated alkylphenols, alkoxylated amines, alkoxylated amides, alkoxylated fatty esters, alkoxylated oils, fatty esters, alkoxylated fatty acids, sorbitan derivatives, and the like.

Typical ionic surfactants found in emulsifiers include alkylaryl sulfonates; alkylarylsulfonic acids; carboxylated alcohol ethoxylates and alkylphenol ethoxylates; carboxylic acids/fatty acids; diphenyl sulfonate derivatives; olefin sulfonates; phosphate esters; phosphorus organic derivatives; quaternary surfactants; sulfates and sulfonates of oils and fatty acids; sulfates and sulfonates of ethoxylated alkylphenols; sulfates of ethoxylated alcohols; sulfates of fatty esters; sulfonates of dodecyl and tridecylbenzenes; sulfonates of naphthalene and alkyl naphthalene; sulfonates of petroleum; sulfosuccinamates; alkanolamides; alkoxylated amines; sarcosine derived surfactants such as, ammonium lauroyl sarcosinate, and the like.

The preferred emulsifiers for this application are alkoxylated alcohols and alkyphenols, preferably of from about 4 to about 50 ethylene oxide units and a hydrophobe of from about 4 to about 16 carbon atoms. Other preferred surfactants are alkylphenol EO/PO block copolymers, e.g., WITCONOL NS 108 LQ, and alkoxylated amine salts of ethoxylated alkylphenol sulfates, e.g., WITCOLATE 1335 from Crompton Corporation, Witco Group.

Developing an effective emulsifier package (blend of emulsifiers) requires careful screening of potential emulsifiers (surfactants), conducting emulsion tests according to prescribed methods, and optimizing the performance of the most promising emulsifier(s). The screening of potential emulsifier packages is dependent upon pesticide active type, and solvents. Those skilled in the art of EC technology understand the importance of HLB and surfactant choice for such formulations. The emulsifier is preferably present at from about 1 to about 50 weight percent of the emulsifiable concentrate, preferably from about 3 to about 10 weight percent.

Solvents

The selection of a suitable solvent system is made by taking into consideration the solubility of the active(s) and the chemical and toxicological profiles of the solvent(s). The solvents include all EPA approved solvents and the list may include: aliphatic paraffinic oils, such as kerosene or refined paraffins; aromatic solvents, such as xylenes; C₉—C₁₂ aromatic solvents, such as AROMATIC 100, 150, AND 200 (Exxon Chemical), ISOPAR L, SOLVESSO 100, 150, and 200; chlorinated hydrocarbons, such as chlorobenzene; alcohols, such as butanol and benzyl alcohol; ketones, such as cyclohexanone and N-methyl pyrollidone; and ethers, such as diethylene glycol and diethoxol. In addition, the solvent may include vegetable oils, methylated vegetable oils, petrol-oils, sugar esters of fatty acids, e.g., sucrose esters, and the like.

The preferred solvents are C₉—C₁₂ hydrocarbon solvents because of their low cost and their ability to solubilize many pesticide toxicants.

The solvent may be present at from 0 to about 95 weight percent of the emulsifiable concentrate, preferably from about 20 to about 50 weight percent.

Optional Ingredients

Other additives may optionally be included in the formulation at levels less than about 5 weight percent. Such additives can include, for example, an antifreeze, pH buffer, a finely divided hydrophobic filler, such as silica or TiO₂, and the like.

Emulsifiable Concentrate Application to Agriculture

Emulsifiable concentrates of the present invention can be diluted with water to a desired solids level and applied onto the targets using any suitable means, such as, for example, spraying, dipping, or rubbing. The amount of active in the final formulation is from about 1 to about 95 weight percent. The amount of antifoam in the EC formulation is present at a concentration sufficient to deliver from about 1 to about 200 ppm to the spray mixture upon dilution to use level.

The advantages and the important features of the present invention will be more apparent from the following examples.

### EXAMPLES

### Manufacture

The organosilicone foam control agents of the present invention can be prepared by the hydrosilation of an SiH intermediate with an allyl polyalkylene oxide, as described in Silicones, Chemistry and Technology (CRC Press 1991), pages 1-6, 75-78, and U.S. Pat. Nos. 3,299,112 and 5,145,879.

Those skilled in the art of organosilicone chemistry will recognize this as a standard procedure for the synthesis of organosilicone copolymers of similar nature to those described herein as part of the present invention.

### Emulsifiable Concentrate Preparation

The preparation of an emulsifiable concentrate containing the silicone antifoam is accomplished by adding the silicone either to the emulsifiers or to the mixtures of emulsifiers, solvent, and active ingredients with proper mixing or blending. Typical ingredients that can be used in the preparation of the emulsifiable concentrates are described above.

### Description of Test Methods

Effect on Foam Control

A shake test was used to measure the foamability of different tank mixtures. Different emulsifiable concentrates with or without silicone antifoam were diluted with water. Therefore, 50 mL of each emulsion was shaken for two minutes with a Wrist shaker. The foam height was recorded 0, 1, 2, and 5 minutes after shaking.

Emulsifiable Concentrates Stability

The stability of the emulsifiable concentrates was examined by storing at room temperature and in a 50° C. oven for three weeks. Samples were observed for appearance (i.e., clarity or phase separation).

### Example 1

The organosilicone foam control agents of the present invention are described in Table 1, along with comparative organomodified siloxanes.

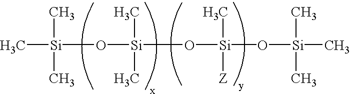

TABLE 1
Description of Organosilicone Foam Control Agents

| | Composition of | | | |
|---|---|---|---|---|
| Reference | Invention | x | y | Description of Z Group |
| Silicone 1 | Yes | 74 | 9 | C₃H₆O(C₃H₆O)₁₃—H |
| Silicone A | No | 7 | 3 | C₃H₆O(C₃H₆O)₁₃—C₄H₉ |
| Silicone B | No | 1 | 0 | C₃H₆O(C₃H₆O)_{1.6}—H |

TABLE 2
Comparative Conventional Antifoams

| Reference | Description |
|---|---|
| Sag 47 (Crompton Corporation, | Dimethylpolysiloxane based antifoam |
| Witco Group) | compound (100%) |
| Silex SE-2 (Exacto) | 20% dimethylpolysiloxane emulsion |

### Example 2

Several model EC formulations were prepared by combining the active ingredient, solvent, emulsifier, and foam control agent together and mixing with a magnetic stir-bar for two minutes to provide the final EC formulation. Tables 3 to 8 describe the various formulations used here to demonstrate the effectiveness of the organosilicone foam control agents of the present invention.

TABLE 3
Butachlor EC Formulation without Antifoam

| Ingredients | Percent by Weight |
|---|---|
| WITCOLATE 1335⁽¹⁾ | 2.8 |
| WITCONOL NS 108 LQ⁽²⁾ | 1.4 |
| AROMATIC 150⁽³⁾ | 1.8 |
| AROMATIC 100⁽⁴⁾ | 37.6 |
| Butachlor | 56.4 |

⁽¹⁾Witcolate 1335 - Alkoxylated amine salts of ethoxylated alkylphenol sulfates (Crompton Corporation, Greenwich, CT).
⁽²⁾WITCONOL NS 108 LQ - Alkylphenol EO/PO block copolymers (Crompton Corporation, Greenwich, CT).
⁽³⁾AROMATIC 150 Solvent - Exxon Chemical Company, Houston, Texas.
⁽⁴⁾AROMATIC 100 Solvent - Exxon Chemical Company, Houston, Texas.

TABLE 4
Butachlor EC Formulation with Silicone 1

| Ingredients | Percent by Weight |
|---|---|
| WITCOLATE 1335 | 2.8 |
| WITCONOL NS 108 LQ | 1.4 |
| AROMATIC 150 | 1.6 |
| Silicone 1 | 0.2 |
| AROMATIC 100 | 37.6 |
| Butachlor | 56.4 |

TABLE 5
Butachlor EC Formulation with Silicone A (Comparative)

| Ingredients | Percent by Weight |
|---|---|
| WITCOLATE 1335 | 2.8 |
| WITCONOL NS 108 LQ | 1.4 |
| AROMATIC 150 | 1.6 |
| Silicone A | 0.2 |
| AROMATIC 100 | 37.6 |
| Butachlor | 56.4 |

TABLE 6
Butachlor EC Formulation with Silicone B (Comparative)

| Ingredients | Percent by Weight |
|---|---|
| WITCOLATE 1335 | 2.8 |
| WITCONOL NS 108 LQ | 1.4 |
| AROMATIC 150 | 1.6 |
| Silicone B | 0.2 |
| AROMATIC 100 | 37.6 |
| Butachlor | 56.4 |

TABLE 7
Butachlor EC Formulation with Sag 47 (Comparative)

| Ingredients | Percent by Weight |
|---|---|
| WITCOLATE 1335 | 2.8 |
| WITCONOL NS 108 LQ | 1.4 |
| AROMATIC 150 | 1.6 |
| Sag 47 | 0.2 |
| AROMATIC 100 | 37.6 |
| Butachlor | 56.4 |

TABLE 8
Butachlor EC Formulation with Silex SE-2 (Comparative)

| Ingredients | Percent by Weight |
|---|---|
| WITCOLATE 1335 | 2.8 |
| WITCONOL NS 108 LQ | 1.4 |
| AROMATIC 150 | 1.6 |
| Silex SE-2 | 0.2 |
| AROMATIC 100 | 37.6 |
| Butachlor | 56.4 |

### Example 3

The compositions of the present invention, when included in a model EC formulation (as described in Example 2) demonstrate superior foam control relative to the comparative organosilicone copolymers, Silicone A, and Silicone B (See Table 9). Although the comparative foam control agents (Sag 47 and Silex SE-2) demonstrate excellent foam control properties, these products do not give a clear homogeneous mixture when incorporated into EC formulations and therefore phase separate. Additionally, the compositions of the present invention not only provide an improvement in foam control, but give a clear stable mixture when incorporated into an EC formulation. The term “stability” as used here indicates that the mixture remains clear and does not phase separate over time.

The comparative organosilicone copolymers (Silicone A and B) do provide clear EC formulations; however, they do not reduce foam relative to the same EC formulation, minus foam control agent (EC without Antifoam)—Table 9.

TABLE 9
Foam Control and Stability Results

| | Foam Height^{a} (mm) | | Stability^{b} | |
|---|---|---|---|---|
| Formulation | 0 Minutes | 1 Minute | RT | 50° C. |
| EC w/o Antifoam | 6 | 6 | Clear | Clear |
| EC w/Silicone 1 | 3 | 0 | Clear | Clear |
| EC w/Silicone B | 6 | 6 | Clear | Clear |
| EC w/Silicone C | 6 | 6 | Clear | Clear |
| EC w/Sag 47 | 0 | 0 | Separated | Separated |
| EC w/Silex SE-2 | 0 | 0 | Separated | Separated |

^{a}Foam height was measured after a two minute shake.
^{b}At both room temperature (RT˜22° C.) and 50° C. oven.

In view of the many changes and modifications that can be made without departing from principles underlying the invention, reference should be made to the appended claims for an understanding of the scope of the protection to be afforded the invention.

What is claimed is:

1. A foam control agent for emulsifiable concentrates comprising a soluble organosilicone of the formula:
(R²(R¹)₂SiO_{1/2})ₑ(SiO_{4/2})_{f}(SiO_{3/2}R¹)_{g}(SiO_{2/2}(R¹)₂)ₓ(SiO_{2/2}R¹Z)_{y}
wherein:
e=2f+g+2;
f=0 to 2;
g=0 to 2;
x=30 to 150;
y=0 to 30;
where, when y>0, x/y=4 to 18 and x+y≧30, and when y=0, the ratio x/e is in the range of from about 20/1 to about 10/1;
R¹=alkyl of 1 to 18 carbon atoms;
R²═R¹ or Z, provided that when y=0, R²═Z;
Z═R³OBₙG;
R³ is a divalent bridging group of two to four carbons;
B is an alkylene oxide group containing two to four carbon atoms, wherein less than ten mole percent of the alkylene oxide functionality is ethylene oxide;
n=8 to30;and
G=hydrogen, a hydrocarbon radical of 1 to 18 carbon atoms, or acetyl.

2. The agent of claim 1 wherein f and g are 0, R₁═R₂, and y is greater than 0.

3. The agent of claim 2 wherein R₁ and R₂ are methyl.

4. The agent of claim 3 wherein Z is C₃H₆O(C₃H₆O)ₙ—H, where n is 8-15.

5. The agent of claim 4 wherein x is 60-100 and y is 4-15.

6. An emulsifiable concentrate comprising:
(A) at least one soluble organosilicone of the formula:
(R²)(R¹)₂SiO_{1/2})ₑ(SiO_{4/2})_{f}(SiO_{3/2}R¹)_{g}(SiO_{2/2}(R¹)₂)ₓ(SiO_{2/2}R¹Z)_{y}
wherein:
e=2f+g+2;
f=0 to 2;
g=0 to 2;
x=30 to 150;
y=0 to 30;
where, when y>0, x/y=4 to 18 and x+y≧30, and when y=0, the ratio x/e is in the range of from about 20/1 to about 10/1;
R¹=alkyl of 1 to 18 carbon atoms;
R²═R¹ or Z, provided that when y=0, R²═Z;
Z═R³OBₙG;
R³ is a divalent bridging group of two to four carbons;
B is an alkylene oxide group containing two to four carbon atoms, wherein less than ten mole percent of the alkylene oxide functionality is ethylene oxide;
n=8 to 30; and
G=hydrogen, a hydrocarbon radical of 1 to 18 carbon atoms, or acetyl;
(B) at least one active ingredient; and
(C) at least one emulsifier.

7. The concentrate of claim 6 wherein f and g are 0, R₁═R₂, and y is greater than 0.

8. The concentrate of claim 7 wherein R₁ and R₂ are methyl.

9. The concentrate of claim 8 wherein Z is C₃H₆O(C₃H₆O)ₙ—H, where n is 8-15.

10. The concentrate of claim 9 wherein x is 60-100 and y is 4-15.

11. The concentrate of claim 6 further comprising an organic solvent.

12. The concentrate of claim 11 wherein the solvent is selected from the group consisting of paraffinic oils, aromatic solvents, chlorinated hydrocarbons, alcohols, ketones, ethers, vegetable oils, petrol oils, and C₉—C₁₂ hydrocarbon solvents.

13. The concentrate of claim 6 wherein the emulsifier is nonionic and is an alkoxylated alcohol or an alkoxylated alkyl phenol.

14. The concentrate of claim 7 wherein the organosilicone is present at from about 0.01 to about 10 weight percent of the concentrate, the emulsifier is present at from about 1 to about 50 weight percent of the concentrate, and the pesticide is present at from about 2 to about 97 weight percent of the concentrate.

15. The concentrate of claim 11 wherein the organic solvent is present at from about 20 to about 50 weight percent of the concentrate.

16. The concentrate of claim 10 further comprising an organic solvent.

17. The concentrate of claim 16 wherein the solvent is selected from the group consisting of paraffinic oils, aromatic solvents, chlorinated hydrocarbons, alcohols, ketones, ethers, vegetable oils, petrol oils, and C₉—C₁₂ hydrocarbon solvents.

18. The concentrate of claim 10 wherein the emulsifier is nonionic and is an alkoxylated alcohol or an alkoxylated alkyl phenol.

19. The concentrate of claim 10 wherein the organosilicone is present at from about 0.01 to about 10 weight percent of the concentrate, the emulsifier is present at from about 1 to about 50 weight percent of the concentrate, and the pesticide is present at from about 2 to about 97 weight percent of the concentrate.

20. The concentrate of claim 16 wherein the organic solvent is present at from about 20 to about 50 weight percent of the concentrate.
